(19) United States
(12) Patent Application Publication
(10) Pub. No.: US 2014/0140940 A1
(43) Pub. Date: May 22, 2014

(54) Cosmetic And Dermatological Photoprotective Preparation With Improved Water Resistance

(71) Applicants: Stefanie Von Thaden, Hamburg (DE); Manuela Koehler, Leon (MX)

(72) Inventors: Stefanie Von Thaden, Hamburg (DE); Manuela Koehler, Leon (MX)

(73) Assignee: BEIERSDORF AG, Hamburg (DE)

(21) Appl. No.: 14/124,096

(22) PCT Filed: Jun. 5, 2012
(86) PCT No.: PCT/EP2012/002370

§ 371 Date: Feb. 4, 2014

(30) Foreign Application Priority Data
Jun. 7, 2011 (DE) 10 2011 077 037.2

(51) Int. Cl. A61K 8/37; A61K 8/06; A61Q 17/04

(52) U.S. Cl. CPC A61K 8/375; A61Q 17/04; A61K 8/062
USPC 424/60; 424/59

(57) ABSTRACT

The invention relates to cosmetic and dermatological photoprotective preparations comprising, in addition to UV filter substances, polyglyceryl-10 stearate as emulsifier. Said preparations have improved water resistance.

The invention relates to water-resistant cosmetic or dermatological photoprotective preparations with improved water resistance on account of the addition or replacement of customary emulsifiers by polyglyceryl-10 stearate.

It is known that as a result of exposure to light with a wavelength in the range from 280 to 400 nm the human epidermis can become tanned and that this is perceived as attractive by people. On the other hand, excessive UV irradiation is harmful to the skin: erythema and skin burns, in colloquial language also sunburn, are the result. Excessive solar irradiation should be avoided or, if this is not possible, be reduced by means of suitable photoprotective products. It is also known that UV-A radiation with a wavelength in the range from 320 to 400 nm causes skin to subsequently develop a tan, but can also bring about a change in the skin, especially in the case of sensitive skin or skin which is continuously exposed to solar radiation. UV-A radiation brings about in particular a loss of elasticity in the skin and the appearance of wrinkles, which leads to premature aging. It favors the triggering of erythema formation and intensifies this reaction in some people and it may even be the cause of toxic or allergic reactions triggered by light. It is therefore desirable to also filter out the UV-A radiation.

In general, the light absorption behavior of photoprotective filter substances is very well known and documented, not least because in most industrialized nations there are positive lists for the use of such substances which apply very strict standards to the documentation. According to in which range the UV light is absorbed, a distinction is made between UV-B filters, UV-A filters and broadband filters, which exhibit a filter effect over the entire UV-A and UV-B range. Through appropriate selection of the UV filter and its concentration in the sunscreen composition, it is possible to influence the degree of shielding of the UV light. However, for the dosing of the substances in the finished formulations, the extinction values can at best offer guidance since imponderables can arise as a result of interactions with ingredients in the formulation or the skin itself. Furthermore, it is generally difficult to estimate beforehand how evenly and in what layer thickness the filter substance is or will be distributed in and on the horny layer of the skin, which is again essential for the protection mechanism.

Besides this influence, the binding capacity of the UV filter in or on the skin is also of great importance for the water resistance of the formulation. It is understandable that oil-soluble UV filters are bonded better to the (lipophilic) surface of the skin and/or are more difficult to wash off from it than water-soluble UV filters.

The effectiveness of sunscreen compositions and/or the UV filters on which they are based is generally determined in biological effectiveness tests under standardized conditions.

The light protection factor LPF, often also called SPF (sun protection factor), indicates the increase in time of solar irradiation permitted by using the sunscreen composition. It is the quotient of erythema threshold time with sunscreen composition and erythema threshold time without sunscreen composition.

The SPF (Sun Protection Factor/light protection factor) is determined in vivo on human skin according to the instructions of the International SPF Test Method (COLIPA, May 2006). At least 10 subjects are required for the test, and the confidence interval for the average value therefrom must not be more than 17%.

Definition is SPF=quotient of minimal erythemal dose (MED) on protected and unprotected skin.

The MED is the lowest dose of UV radiation which after 16-24 h brings about a slight, but clear evident skin reddening (sunburn, erythema). Sources of radiation are sun simulators, mostly xenon lamps.

UV radiation of varying frequency/wavelength penetrates into tissue to differing depths. Consequently, the type of damage is also wavelength-dependent. UV-A protection is of particular importance here since this UV radiation (UV-A (wavelength 320-400 nm) is essentially responsible for skin aging.

UVA filters are types of chemical photoprotective filters. They absorb the energy-rich UV radiation and convert this to heat or light energy which can no longer cause damage.

To test the UV-A protection performance it is possible for example, to use the IPD method (IPD=immediate pigment darkening). In this—similarly to the determination of the light protection factor—a value is determined which indicates how much longer the skin protected with the sunscreen composition can be irradiated with UV-A radiation until the same pigmentation occurs as for the unprotected skin.

Another test method, established Europe-wide, is the Australian standard AS/NZS 2604:1997. This measures the absorption of the preparation in the UV-A region. In order to satisfy the standard, the preparation has to absorb at least 90% of the UV-A radiation in the range 320-360 nm.

In order to give information about the UVA protection performance of a sunscreen product, various test models, both in vivo and also in vitro, can be used, and statements derived therefrom can be made on the packagings (UVA protection according to the Australian standard, Persistent Darkening Factor etc.). However, these details cannot be compared with one another. For this reason, on Sep. 22, 2006, the EU Commission published a recommendation intended to provide more safety and transparency with regard to sunscreen compositions. The EU recommendation envisages a UVA protection of 1/3 in the ratio to the UVB protection. Hitherto, only the protection of sunburn-causing UVB rays was regulated in the cosmetics ordinance. With the new EU recommendation, every sunscreen composition should also simultaneously protect against UVA radiation. The consumer is to assume that UVA protection is ensured which increases with increasing SPF value. For ethical reasons, an in vitro method is proposed for this purpose. At Beiersdorf AG in Hamburg an in vitro method for UVA determination as a function of the light protection factor was developed: the UVA/UVB balance. In February 2005, the UVA/UVB balance was published as DIN 67502 and is therefore the first official method for determining UVA protection in the European region. The method for measuring the UVA protection performance is also revised by COLIPA because the industry requires a uniform method. COLIPA is the umbrella association for the European cosmetics industry, giving inter alia recommendations as regards carrying out the measurement method. The UVA/UVB balance constitutes the basis of the method discussed therein. It is modified in that it is expanded by a pre-irradiation with UV light in order to be able to also include a number of special UV filter systems. Hitherto, the products were tested without pre-irradiation. As a result, no information could be given as to whether the protective effect of the product is also present under real conditions as a result of the effect of sun. This expanded measurement method carries the name “COLIPA Ratio”.

This means that UVA filters are used in the ratio of 1:3 to UVB filters and consequently the UVA protection is increased with increasing light protection. The EU recommendation envisages a UVA protection of 1/3 in the ratio to the UVB protection. This measurement method carries the name “COLIPA Ratio”. The COLIPA ratio must, according to the formula, be

$$\frac{UVB}{UVA} < 3.$$

The COLIPA ratio is the standard for determining UVA protection in Europe.

As regards stabilization and attainment of high UV protection, there are a multitude of disclosures in the prior art.

Frequent types of cosmetic or dermatological preparations are finely disperse multiphase systems in which one or more fatty or oil phases are present besides one or more water phases. Among these systems, the actual emulsions are in turn most widespread.

However, low-emulsifier or emulsifier-free preparations based on so-called hydrodispersions are also known. Hydrodispersions are dispersions of a liquid, semisolid or solid internal (discontinuous) lipid phase in an external aqueous (continuous) phase. Hydrodispersions—like emulsions which are characterized by a similar phase arrangement—are metastable systems and therefore have a tendency to convert to a state of two interconnected discrete phases. In a classic O/W emulsion, the choice of a suitable emulsifier prevents phase separation. In contrast to the classic emulsions, hydrodispersions, however, comprise only very small amounts of emulsifiers and can even be entirely free from emulsifiers.

Cosmetic or medical preparations that are often used are emulsions, in particular W/O, O/W, O/W/O or W/O/W emulsions. In general, emulsions are understood as meaning heterogeneous systems which consist of two liquids that are immiscible or have only limited miscibility with one another, these usually being referred to as phases. In an emulsion, one of the two liquids (W/O) is dispersed in the form of very fine droplets in the other liquid. The liquids (pure or in the form of solutions) are present in an emulsion in a more or less fine distribution, which is generally only of limited stability.

If the two liquids are water and oil and oil droplets are finely distributed in water, then this is an oil-in-water emulsion (O/W emulsion, e.g. milk). The basic character, for example electrical conductivity, sensory properties, ability of the continuous phase to be dyed, of an O/W emulsion is determined by the water. In the case of a water-in-oil emulsion (W/O emulsion, e.g. butter), the principle is reversed, the basic character here being determined by the oil.

The prior art is aware of several essential factors which have a positive influence on the stability and rheology of emulsions.

For their formation and stabilization, emulsions generally require one or more emulsifiers, thickeners and/or consistency regulators in order to be stable over a cosmetically acceptable period, in general one year after opening a cosmetic preparation.

For this, large amounts of emulsifiers are often required. This in turn can lead for consumers to incorrect sensations ranging to incompatibility and in extreme cases even phenomena such as “Mallorca acne” or the like.

It is therefore desirable to provide emulsion preparations which comprise extremely small amounts of emulsifiers but are nevertheless formulated to have adequate stability.

The water resistance of photoprotective preparations is a further challenge placed on the person skilled in the art of cosmetics. The water resistance of a photoprotective product is essential. Particularly at the beach, one does not wish to have to reapply cream after every swim in the sea or after every shower.

O/W emulsions can be spread easily and are also suitable for greasy skin and acne. A disadvantage is often the low water resistance, especially for formulations which are easily absorbed.

W/O emulsions are highly suitable for dry skin, have high water resistance and can be readily combined with pigments. However, they are sticky, are absorbed less well and are therefore not liked by some users.

Hydrodispersion gels can be spread easily and lead to a pleasant skin feel. They can be produced without emulsifiers and preservatives and are therefore readily suitable for people with sun allergy. They are suitable for hairy areas of the skin and for greasy skin and acne, but have low water resistance.

Although sunscreen compositions in sprays can be spread easily, they mostly have a low water resistance.

Water resistant photoprotective formulations are usually achieved using one or more of the film formers customary in photoprotective formulations (inter alia Unimer or Antaron grades, and synchrowaxes). However, these systems are characterized by sensory properties that are unattractive to the consumer, i.e. they are often particularly dull, sticky and oily on the skin and are not absorbed very well.

A further object of the invention is to improve this situation.

There are many emulsifiers with which the water resistance is not adversely affected. For example the fatty acid esters, such as glyceryl stearate citrate (Imwitor® from Sassol), do not adversely affect the water resistance. As explained above, however, all emulsifiers influence the sensory properties of photoprotective preparations. Emulsifiers which have a positive effect on the water resistance tend to be lipophilic and the resulting sensory properties is somewhat rich, heavy and/or sticky. If the person skilled in the art, however, turns to hydrophilic emulsifiers, the sensory properties become light and nonsticky. These sensory properties are preferred by consumers.

A problem here, however, is that hydrophilic emulsifiers adversely affect the water resistance because the hydrophilic emulsifier is readily washed off on account of its hydrophilicity and, in so doing, in its property as an emulsifier, takes with it the UV filters. The properties “light formulation by means of hydrophilic emulsifier” and “poor water resistance by means of hydrophilic emulsifier” do not appear to be in agreement with the consumer wish for a light and water resistant formula. This appears to be a quasi unsolvable problem, with a decision having to be made between light sensory properties and water resistance.

Furthermore, cosmetic or dermatological preparations must satisfy a number of esthetic aspects in order to gain adequate consumer acceptance.

Emulsifiers from the food sector are known to the person skilled in the art, such as glyceryl stearate citrate (Imwitor®) or polyglyceryl-10 stearate.

In the search for suitable emulsifiers which correspond to the desired requirements of cosmetics and dermatology, however, these emulsifiers appear to exclude themselves.

Glyceryl stearate citrate has to be used in cosmetic preparations in such a high amount or in combination with coemulsifiers in order to obtain stable emulsions that the desire for a reduced emulsifier content cannot be fulfilled with glyceryl stearate citrate.

Polyglyceryl-10 stearate (PG-10 stearate, CAS No.: 79777-30-3) is a hydrophilic emulsifier and has an HLB of 12.

Commercially, polyglyceryl-10 stearates in the form of mixtures are obtainable for example as Polyaldo-10-1-S (Lonza), Nikkol Decaglyn 1-S (Nikko Chemicals Co., Ltd.) or Salacos PGMSV (The Nisshin OilliO Group, Ltd.)

Polyglyceryl-10 stearate is known from the production of ice cream and is brown in color. Also on account of this esthetically problematic circumstance, use in cosmetic emulsion appeared to be naturally ruled out.

Heliogel®, which also comprises polyglyceryl-10 stearate besides sodium acrylates copolymer, hydrogenated polyisobutene, phospholipids, Helianthus Annuus (sunflower) seed oil, is commercially available.

In cosmetic preparations, PG-10 stearate is merely named as a constituent of an aftershave gel. In this preparation, 6% by weight of PG-10 stearate as well as other emulsifiers such as lecithin and PEG 150 distearate are described (WO 2003082182).

JP 2005162664 discloses PG-10 stearate in a hair coloring preparation comprising peroxides. A cosmetic topical application is therefore ruled out.

U.S. Pat. No. 5,925,615 discloses PG-10 stearate in shampoo formulations comprising polyethylene glycols and parabens.

DE 19547679 A1 describes a haircare emulsions with PG-10 stearate.

DE 19724587 A1 describes a hair rinse with PG-10 stearate, and polyethylene glycols and parabens.

WO 0027197 A1 discloses preparations comprising, apart from PG-10 stearate, one or more UV filters, lecithins, polyethylene glycols and parabens.

DE 102008028821 A1 and DE 102008028822 A1 disclose stick preparations with PG-10 stearate.

DE 10213955 A1 discloses PG-10 stearate in an aftershave lotion, where the content of PG-10 stearate is 6% and more.

US 20060018853 describes a peel-off preparation with PG-10 stearate with decaglyceryl triisostearate and lecithin.

Surprisingly, however, it was found that polyglyceryl-10 stearate can be used as an emulsifier in cosmetic or dermatological preparations as emulsifier without exhibiting the disadvantages concerning the coloring effect and the instability.

By adding PG-10 stearate instead of other emulsifiers, the water resistance is also surprisingly improved here. This is all the more surprising since polyglyceryl-10 stearate (PG-10 stearate) is a hydrophilic emulsifier.

It is particularly surprising that PG-10 stearate can be used either as the sole emulsifier or in emulsifier mixtures and the photoprotective preparations exhibit improved water resistance.

The invention is therefore a water resistant cosmetic or dermatological photoprotective preparation comprising one or more UV light filter substances and polyglyceryl-10 stearate.

The fraction of polyglyceryl-10 stearate in the photoprotective preparation is to be selected as less than 6% by weight, based on the total mass of the preparation. Particularly preferably, the fraction of PG-10 stearate is to be selected as less than 3% by weight, in particular in the range from 0.1 to 2.8% by weight, based on the total mass of the preparation. The advantage here is that the wish for low emulsifier contents is satisfied.

Excluded from the preparations according to the invention are O/W emulsion preparations 4 and 5 consisting of

| | 4 | 5 |
|---|---|---|
| Panthenol | 0.7 | 0 |
| Butylene glycol | 5 | 10 |
| Propylene glycol | 5 | — |
| Benzethonium chloride | 0.5 | 0.9 |
| Lauroyl ethyl arginate | 1.0 | 0.5 |
| Medicinal white oil | 0 | 0 |
| Isopropyl palmitate | 0 | 0 |
| Caprylic/capric triglyceride | 0 | 1.00 |
| Cetearyl alcohol | 2.5 | 4.00 |
| Cetyl alcohol | 0 | 0 |
| Linear silicone oil | 0 | 0.50 |
| Cyclic silicone oil | 0 | 0 |
| C12-15 Alkyl benzoate | 0 | 2.00 |
| Shea butter | 0 | 0 |
| Dicapryl ether | 10 | 0 |
| Dicapryl carbonate | 0 | 0 |
| Glyceryl stearate | 2.4 | 2.4 |
| Tapioca starch | 0 | 0 |
| Glycerol | 8 | 8 |
| Butylene glycol | 1 | 0 |
| Citric acid | 0 | 0 |
| Sodium hydroxide solution 45% | 0.25 | 1 |
| Polyglyceryl-10 myristate | 0 | 0 |
| Polyglyceryl-10 stearate | 1 | 1 |
| Polyacrylic acid, Na salt (Carbopol 981) | 0 | 0 |
| Acrylic acid/VP crosspolymer | 0.5 | 0.75 |
| Trisodium EDTA | | 1 |
| Ethylhexyl methoxycinnamate | 2 | 0 |
| Butylmethoxydibenzoylmethane | 0 | 2 |
| Phenylbenzimidazolsulfonic acid, sodium salt | 0 | 2 |
| Ethylhexyl salicylate | 0 | 2 |
| Titanium dioxide, silicone-coated | 0.3600 | 0 |
| Octocrylene | 0 | 2 |
| Perfume | 0 | 0.20 |
| Water | ad 100 | ad 100 |

and O/W emulsion preparations 49 and 50 consisting of

| | 49 | 50 |
|---|---|---|
| Panthenol | 0.7 | 0 |
| Butylene glycol | 5 | 10 |
| Propylene glycol | 5 | — |
| Methylisothiazolinones | 0.06 | — |
| Benzethonium chloride | 0.15 | — |
| Piroctone olamine | 0.15 | — |
| Lauroyl ethyl arginate | 0.15 | 1.75 |
| Medicinal white oil | 0 | 0 |
| Isopropyl palmitate | 0 | 0 |
| Caprylic/capric triglyceride | 0 | 1.00 |
| Cetearyl alcohol | 2.5 | 4.00 |
| Cetyl alcohol | 0 | 0 |
| Linear silicone oil | 0 | 0.50 |
| Cyclic silicone oil | 0 | 0 |
| C12-15 Alkyl benzoate | 0 | 2.00 |
| Shea butter | 0 | 0 |
| Dicapryl ether | 10 | 0 |
| Dicapryl carbonate | 0 | 0 |
| Glyceryl stearate | 2.4 | 2.4 |
| Tapioca starch | 0 | 0 |
| Glycerol | 8 | 8 |
| Citric acid | 0 | 0 |
| Sodium hydroxide solution 45% strength | 0.25 | 1 |
| Polyglyceryl-10 myristate | 0 | 0 |
| Polyglyceryl-10 stearate | 1 | 1 |
| Polyacrylic acid, Na salt | 0 | 0 |
| Acrylic acid/VP crosspolymer | 0.5 | 0.75 |
| Trisodium EDTA | | 1 |
| Ethylhexyl methoxycinnamate | 2 | 0 |
| Butylmethoxydibenzoylmethane | 0 | 2 |
| Phenylbenzimidazolsulfonic acid | 0 | 2 |
| Ethylhexyl salicylate | 0 | 2 |
| Titanium dioxide + trimethoxycaprylsilane | 0.3600 | 0 |
| Octocrylene | 0 | 2 |
| Perfume | 0 | 0.20 |
| Water | ad 100 | ad 100 |

The numerical values are fractions by weight based on the total mass of the preparations.

The preparations according to the invention are advantageously based on an emulsion, preferably an O/W emulsion, or a hydrodispersion. The preparations produced in this way, preferably emulsions, have a low emulsifier content of less than 6% and therefore satisfy the wish for readily compatible, mild products without the stability being impaired. Less than 6% by weight or less than 3% by weight of PG-10 stearate does not mean 0% by weight. Preferably, the minimum fraction of PG-10 stearate is 0.1% by weight. Surprisingly, the preparations produced with less than 6% by weight of polyglyceryl-10 stearate are stable for a period of at least 3 years at room temperature and for at least 6 months when stored at 40° C.

Furthermore, it was surprisingly found that polyglyceryl-10 stearate leads to stable emulsions even if used as the only emulsifier. Dispensing with further additional emulsifiers is therefore possible, thus complying with the wish for reduced emulsifier contents.

Film formers are known as substances for achieving the water resistance of preparations. These are characterized by the following property: if a film former is dissolved in water or other suitable solvents and the solution is then applied to the skin, then following the evaporation of the solvent a film is formed which essentially serves to fix the photoprotective filter substances on the skin and in particular to ensure and/or to increase the water resistance of the preparation on the skin.

Known film formers are, for example, polymers based on PVP (Antaron V216, V220 from GAF Chemicals Cooperation), sodium polystyrenesulfonate (Flexan 130 from National Starch) or polyisobutene (Rewopal PIB 1000). Further film formers that are typical for photoprotective products and improve the water resistance are, for example, Unimer or Antaron grades and synchrowaxes.

Compared with preparations without PG-10 stearate but with the same UV filter fraction and film formers, the preparations according to the invention have improved water resistance.

The water resistance of sunscreen products can be determined according to the instructions of the COLIPA WR (water resistance) method from December 2005 in vivo on human skin or via the contact angle method.

The SPF determination takes place once on dry skin (static) and a second time after watering for at least 2×20 minutes and a drying time of 15 min (2×20 min=water resistant, 4×20 min=extra water resistant). The water resistance is confirmed if the value after watering after deducting the confidence interval is greater than 50% of the SPF static.

Apart from this in vivo test there is also an in vitro test via the contact angle method (Contact angle measurement-a reliable supportive method for screening water-resistance of ultraviolet-protecting products in vivo” Intern. Journal of Cosmetic Science, 2007, 29, 283-291).

The preparations in Table 1 were investigated in accordance with this method.

TABLE 1
Water resistance of cosmetic photoprotective preparations

| INCI | 1 | 2 |
|---|---|---|
| Xanthan Gum | 0.4000 | 0.4000 |
| Tocopheryl Acetate | 0.0600 | 0.0600 |
| Cetyl Alcohol | 2.2000 | — |
| Octyldodecanol | 5.5000 | 5.5000 |
| Myristyl Myristate | 1.5000 | 1.5000 |
| C12-15 Alkyl Benzoate | 6.5000 | 6.5000 |
| Butylene Glycol Dicaprylate/Dicaprate | 2.5000 | 2.5000 |
| Cetearyl Alcohol | — | 2.5000 |
| Cetearyl Alcohol + PEG-40 Castor Oil + Sodium | 2.0000 | — |
| Cetearyl Sulfate | | |
| Glyceryl Stearate SE | 0.6500 | — |
| Glyceryl Stearate | — | 0.8500 |
| Tapioca Starch + Aqua | 1.0000 | 1.0000 |
| Parfum | 0.4000 | 0.4000 |
| Glycerin | 0.9000 | 0.9000 |
| Sodium Hydroxide aq | 0.6000 | 0.6000 |
| Polyglyceryl-10-Stearate | — | 2.5500 |
| Phenoxyethanol | 0.2000 | 0.2000 |
| Methylparaben | 0.3000 | 0.3000 |
| Acrylates/C10-30 Alkyl Acrylate Crosspolymer | 0.0500 | 0.0500 |
| Aqua | Ad 100 | Ad 100 |
| Alcohol Denat. | 8.0000 | 8.0000 |
| Aqua + Trisodium EDTA | 1.0000 | 1.0000 |
| Ethylhexyl Methoxycinnamate + BHT | 0.5000 | 0.5000 |
| Octocrylene | 4.5000 | 4.5000 |
| Bis-Ethylhexyloxyphenol Methoxyphenyl Triazine | 3.5000 | 3.5000 |
| Butyl Methoxydibenzoylmethane | 4.5000 | 4.5000 |
| Titanium Dioxide + Trimethoxycaprylylsilane | 4.0000 | 4.0000 |
| Phenylbenzimidazole Sulfonic Acid | 2.0000 | 2.0000 |
| Diethylhexyl Butamido Triazone | 1.0000 | 1.0000 |
| Contact angle | 24.8 | 61.0 |

By replacing the emulsifiers cetyl alcohol and cetearyl alcohol (PEG 40 castor oil, sodium cetearyl sulfate) with PG-10 stearate, the contact angle is increased from 24.8° to 61°.

In the literature, the contact angle is given as an indicator for the water resistance of a cosmetic formulation. It is assumed that an angle >35° is an indicator for a water-resistant formula. Water-resistant preparations according to the invention accordingly have a contact angle greater than 35° (contact angle method).

Consequently, by replacing the emulsifier system, a water-resistant formula is obtained according to the invention for the first time. Such an effect can normally only be achieved by means of film formers, but with all of the undesired disadvantages already discussed. This can now be avoided according to the invention.

The investigations reveal in formulas 1 and 2 carbomer and xanthan gum only for adjusting the viscosity, i.e. no classic film formers. The water-resistance-improved PG-10 stearate formula 2 likewise comprises no film formers. The water resistance was thus merely achieved by replacing the emulsifier system.

Polyglyceryl-10 stearate can therefore be used as emulsifier in light protection preparations comprising one or more UV filter substances and optionally one or more film formers for improving the water resistance compared with the same preparations without polyglyceryl-10 stearate.

The preparations according to the invention advantageously comprise one or more film formers for further adjusting the water resistance.

Furthermore, it has surprisingly been found that PG-10 stearate can be added either to the water phase or to the fatty phase during the emulsion formation. The same product is obtained in both cases. The fluctuations in the consistency are insignificant and irrelevant for a consumer.

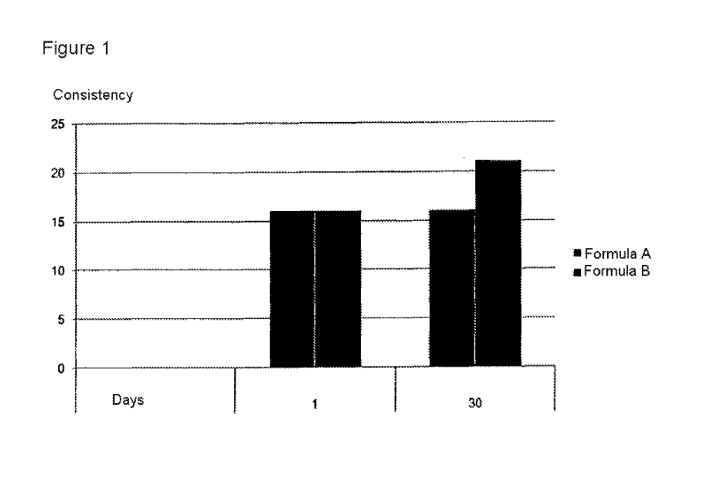

FIG. 1 shows these consistency investigations. The consistency was determined using a consistency determining instrument according to Fligge (according to DE 29 09 087).

On the X axis is plotted firstly the value of the consistency of the formulae A and B to be compared after one day (left) and after 30 days (right). Formula A differs from formula B in that the emulsifier PG-10 stearate was added to the water phase in the case of A and to the fatty phase in the case of B. All of the other steps were identical for both formulations.

The consistency value is plotted on the y axis. It can be seen that after 1 day both formulae even have the same consistency in numerical terms, i.e. are identical with regard to consistency. After 30 days, although there is a numerical difference, it is not perceivable by the consumer.

The investigations demonstrate the extraordinary properties of PG-10 stearate. Since it is irrelevant to which phase the emulsifier is added, one has greater flexibility in phase division during production.

A process for producing cosmetic or dermatological emulsion preparations with polyglyceryl-10 stearate as emulsifier, preferably the only emulsifier, permits addition both to the water phase and/or to the oil phase of the emulsion.

Preparations containing PG-10 stearate can thus be produced more easily and give the person skilled in the art exceptional flexibility as regards viscosity, sensory properties, application form and tolerance towards a very wide variety of ingredients and the production.

To limit the use of PG-10 stearate in cosmetics to the lowest possible fractions may have been obvious to a person skilled in the art of cosmetics on account of the brown color of the PG-10 stearate, but said person would then also have to accept a reduced emulsifying effect on account of the small fractions. However, this has surprisingly not proven to be true when using less than 6% by weight of PG-10 stearate, in particular in the range from 0.1 to 2.8% by weight, based on the total amount of the preparation.

In several long-term stability investigations, the PG-10-stearate-containing preparations exhibited no instabilities, phase separation, coalescence, Ostwald ripening or change in droplet size over time or creaming. The formulations, as disclosed in the examples, were optically stable for at least 6 months at room temperature and also at 40° C. This means no phase separation or phase deposition was observed.

Besides the improved water resistance, the preparations according to the invention have, in contrast to the prior art, good product sensory properties and the UV light protection performance is not limited.

Additional emulsifiers can be added to the preparations according to the invention. However, this is not necessary or obligatory in all cases according to the invention. If additional emulsifiers are added, these should preferably be selected from the group of emulsifiers which are solid, pasty or liquid at 25° C. and not ethoxylated. Particularly preferred additional emulsifiers should be selected from the group 1,2-propanediol fatty acid esters, acetoglycerides, acetylated mono/diglycerides, alkali metal and ammonium soaps, ammonium phosphatides, sorbitan monoisostearate, C10-C22 fatty acids, cetearyl sulfate, cetearyl glucosides, cetyl phosphate, cetylstearyl alcohol in combination with sodium cetylstearyl sulfate, citroglyceride esters, citric acid monoglycerides, diacetyltartaric acid monoglycerides, diisostearoylpolyglyceryl-3 diisostearate (Isolan PDI), egg lecithin, emulsifier YN (trade name), acetic acid monoglycerides, mixed propylene glycolgl ycerol esters, glyceryl laurate, glyceryl myristate, glyceryl oleate in combination with propylene glycol, glyceryl stearate, glyceryl stearate SE, glyceryl stearate citrate, glycol distearate, isostearyl diglyceryl succinate, isostearyl glyceryl ether, potassium cetyl phosphate, lactoglycerides, lactylated mono/diglycerides, lecithin, methylglucose sesquistearate, lactic acid monoglycerides, mono- and diglycerides of food fatty acids esterified with acetic acid and tartaric acid, monoglyceride acetate, monoglyceride citrate, monoglyceride diacetyltartrate, monoglyceride lactate, monoglyceride tartrate, Na, K and Ca salts of stearoyl-2-lactyllactic acid, Na, K and Ca stearoyl-2-lactoyllactate, Na, K and Ca stearoyl-2-lactyllactate, Na, K and Ca stearoyllactic acid, Na salt of laurylsulfuric acid, sodium cetyl phosphate, sodium cetylstearyl sulfate, sodium dodecylsulfate and/or dodecylhydrogensulfate, polyglycerol-3 diisostearate (Lameform TGI), polyglycerol esters of interesterified ricinoleic acid, polyglycerol fatty acid esters, polyglcyerol polyricinoleate, polyglycerol dimerate isostearate, polyglyceryl polyricinoleate (Admul WOL 1403), polyglyceryl-1 dipolyhydroxystearate (Dehymuls PGPH), polyglyceryl-2 laurate, polyglyceryl-2 sesquiisostearate, polyglyceryl-3 beeswax (Cera Bellina), polyglyceryl-3 cetyl ether (Chimexane NL), polyglyceryl-3 distearate (Cremophor GS 32), polyglyceryl-3 methylglucosedistearate (Tego Care 450), polyglyceryl-3 oleate, polyglyceryl-3 methyl-glycosedistearate, polyglyceryl-4 caprate (polyglycerol caprate T2010190), polyglyceryl-4 diisostearate/poly-hydroxystearate/sebacate (Isolan GPS), polyglyceryl-4 isostearate (Isolan GI 34), polyoxyethylenestearic acid esters, polyoxystearate mono/diglyceride, propylene glycolstearate SE, propylene glycol fatty acid esters, propylene glycol mono/di-fatty acid esters, rapeseed lecithin, saccharoglycerides, saccharose esters of food fatty acids, soya lecithin, sorbates e.g. sorbitan monolaurate (Sorbate 20), sorbitan dicitrate, sorbitan dierucate, sorbitan dihydroxystearate, sorbitan diisostearate, sorbitan dimaleate, sorbitan dioleate, sorbitan diricinoleate, sorbitan ditartrate, sorbitan monocitrate, sorbitan monoerucate, sorbitan monohydroxystearate, sorbitan monomaleate, sorbitan monoricinoleate, sorbitan monostearate (Sorbate 60), sorbitan monotartrate, sorbitan sesquicitrate, sorbitan sesquierucate, sorbitan sesquihydroxystearate, sorbitan sesquiisostearate, sorbitan sesquimaleate, sorbitan sesquioleate, sorbitan sesquiricinoleate, sorbitan sesquitartrate, sorbitan tricitrate, sorbitan trierucate, sorbitan trihydroxystearate, sorbitan triisostearate, sorbitan monooleate, sorbitan trismaleate, sorbitan trioleate, sorbitan triricinoleate, sorbitan tristearate (sorbate 65), sorbitan tritartrate, stearic acid and its salts, sucro esters, triethylcitrate, tartaric acid glycerides, tartaric acid monoglycerides and/or sugar fatty acid esters, particular preference being given to glyceryl stearate.

Preferably, the preparations according to the invention comprise no further additional emulsifier apart from PG-10 stearate. The fraction of additional emulsifiers should thus be preferably less than 0.01% by weight, based on the total mass of the preparation, in order to be classed as in accordance with the invention—without additional emulsifier.

Lecithins is the classic name for a group of chemical compounds, the so-called phosphatidylcholines. These are phospholipids which are composed of fatty acids, glycerol, phosphoric acid and choline. Lecithins are constituents of the cell membrane of animal and plant life-forms. They are accompanying substances in fats and oils. They permit the emulsification of fats and water and are therefore important natural surfactants (emulsifiers) for foods and feeds. Lecithins are generally approved in the EU as food additive (E 322) for foods with a maximum quantitative limit exclusively for baby food. In medicine and in cosmetics, they are also used as an active ingredient, and in dietetics as a food supplement. Besides their structure-forming properties, numerous functional tasks are attributed to the lecithins. They are actively involved both in anabolic lipid metabolism and also in catabolic fat metabolism.

Lecithins are therefore to be excluded according to the invention.

According to the invention, it is advantageous if the preparation comprises UV filters in the fatty phase and/or in the aqueous phase. In this connection, it is preferred according to the invention if the UV filters selected are one or more compounds from the group 1-(4′-tert-butylphenyl)-3-(4′-methoxyphenyl)propan-1,3-dione, 1,4-di(2-oxo-10-sulfo-3-bornylidenemethyl)benzene and salts thereof, 1-phenyl-3-(4′-isopropylphenyl)propan-1,3-dione, 2-(2H-benzotriazol-2-yl)-4-methyl-6-[2-methyl-3-[1,3,3,3-tetramethyl-1-[(trimethylsilyl)oxy]disiloxanyl]propyl]phenol, hexyl 2-(4′-diethylamino-2′-hydroxybenzoyl)benzoate, 2,2′-methylenebis(6-(2H-benzotriazol-2-yl)-4-(1,1,3,3-tetra-methylbutyl)phenol, 2,2′-dihydroxy-4-methoxybenzophenone, 2,4,6-tris(biphenyl)-1,3,5-triazine, 2,4-bis(4′-dineopentylaminobenzalmalonate)-6-(4″-butylaminobenzoate)-s-triazine, 2,4-bis[5-1(dimethylpropyl)benzoxazol-2-yl-(4-phenyl)imino]-6-(2-ethylhexyl)imino-1,3,5-trazine with the (CAS No. 288254-16-0), 2,4-bis{[4-(1′,1′,1′,3′,5′,5′,5′-heptamethylsiloxy-2-methylpropyloxy)-2-hydroxyl]phenyl}-6-(4-methoxyphenyl)-1,3,5-triazine, 2,4-bis{[4-(2-ethylhexyloxy)-2-hydroxyl]-phenyl}-6-(1-methylpyrrol-2-yl)-1,3,5-triazine, 2,4-bis{[4-(2-ethylhexyloxy)-2-hydroxyl]phenyl}-6-(4-methoxyphenyl)-1,3,5-triazine (INCI: Bis-Ethylhexyloxyphenol Methoxyphenyl Triazine), 2,4-bis{[4-(2-ethylhexyloxy)-2-hydroxyl]phenyl}-6-[4-(2-methoxyethylcarboxyl)phenylamino]-1,3,5-triazine, 2,4-bis{[4-(2-methylpropenyloxy)-2-hydroxyl]-phenyl}-6-(4-methoxyphenyl)-1,3,5-triazine, 2,4-bis{[4-(3-(2-propyloxy)-2-hydroxypropyloxy)-2-hydroxyl]phenyl}-6-(4-methoxyphenyl)-1,3,5-triazine, 2,4-bis{[4-(3-(2-propyloxy)-2-hydroxypropyloxy)-2-hydroxy}phenyl}-6-[4-(ethylcarboxyl)phenylamino]-1,3,5-triazine, 2,4-bis{[4-(3-sulfonato)-2-hydroxypropyloxy)-2-hydroxy]phenyl}-6-(4-methoxyphenyl)-1,3,5-triazine sodium salt, 2,4-bis{[4-tris(trimethylsiloxysilylpropyloxy)-2-hydroxy]phenyl}-6-(4-methoxyphenyl)-1,3,5-triazine, 2-ethylhexyl methoxycinnamate, 2-ethylhexyl-2-cyano-3,3,-diphenylacrylate (octocrylene), 2-ethylhexyl 2-hydroxybenzoate, 2-hydroxy-4-methoxy-4′-methylbenzophenone, 2-hydroxy-4-methoxybenzophenone, 2-methyl-5-(2-oxo-3-bornylidenemethyl)sulfonic acid and salts thereof, 2-phenylbenzimidazole-5-sulfonic acid salts, 3-(4-(2,2-bis-ethoxycarbonylvinyl)phenoxy)propenyl)methoxysiloxane/dimethylsiloxane copolymer, 3-(4-methylbenzylidene)camphor, 3-benzylidenecamphor, 4-(2-oxo-3-bornylidenemethyl)-benzenesulfonic acid, 4-(2-oxo-3-bornylidenemethyl)benzenesulfonic acid salts, 2-ethylhexyl 4-(dimethylamino)benzoate, amyl 4-(dimethylamino)benzoate, 4-(tert-butyl)-4′-methoxydibenzoylmethane, tris(2-ethylhexyl) 4,4′,4″-(1,3,5-triazine-2,4,6-triyltriimino)trisbenzoate (also: 2,4,6-tris[aniline(p-carbo-2′-ethyl-1′-hexyloxy)]-1,3,5-triazine (INCI: Ethylhexyl Triazone), 4-dicyanomethylene-2,6-dimethyl-1,4-di-hydropyridine-N-(ethyloxysulfate ester salt), 4-dicyanomethylene-2,6-dimethyl-1,4-dihydropyridine-N-(ethyloxysulfate ester salt) the monosodium salt, 4-isopropylbenzyl salicylate, di(2-ethylhexyl) 4-methoxybenzalmalonate, 2-ethylhexyl 4-methoxycinnamate, isoamyl 4-methoxycinnamate, benzene-1,4-di(2-oxo-3-bornylidenemethyl-10-sulfonic acid), benzophenone-3, benzophenone-4, bis-ethylhexyloxyphenol methoxyphenol triazine (Tinosorb® S), bisoctrizole, butylmethoxydibenzoylmethane, diethylaminohydroxybenzoyl hexylbenzoate, dimethicodiethyl benzalmalonate, dioctylbutylamidotriazone (INCI: Diethylhexyl-Butamidotriazone), disodium phenyldibenzimidazoletetrasulfonate, ethylhexylmethoxycrylene, ethylhexylsalicylate (octylsalicylate), ethylhexyl salicylate, homomethyl salicylate, homosalate, isoamyl p-methoxycinnamate, polysilicone-15, merocyanine, methylenebis-benzotriazolyltetramethylbutylphenol, titanium dioxide (with and without coating), phenylbenzimidazolesulfonic acid, phenylene-1,4-bis(2-benzimidazyl)-3,3′-5,5′-tetrasulfonic acid salts, piperazine derivatives, polysilicone-15, 2-ethylhexyl salicylate, 4-isopropylbenzyl salicylate, homomethyl salicylate, terephthalidenedicamphorsulfonic acid, terephthalidenecamphor sulfonic acid, titanium dioxide and/or zinc oxide.

For the purposes of the present invention, the pigments (titanium dioxide, zinc oxide) can also advantageously be used in the form of commercially available oily or aqueous predispersions. Dispersion auxiliaries and/or solubilizers can advantageously be added to these predispersions.

According to the invention, the pigments (titanium dioxide, zinc oxide) can advantageously be surface-treated (“coated”), the intention being, for example, to form and/or retain a hydrophilic, amphiphilic or hydrophobic character. This surface treatment can consist in providing the pigments with a thin hydrophilic and/or hydrophobic inorganic and/or organic layer by processes known per se. Within the context of the present invention, the various surface coatings can also comprise water.

In the context of the present invention, inorganic surface coatings can consist of aluminum oxide (Al₂O₃), aluminum hydroxide Al(OH)₃, or aluminum oxide hydrate (also: alumina, CAS No.: 1333-84-2), sodium hexametaphosphate (NaPO₃)₆, sodium metaphosphate (NaPO₃)ₘ, silicon dioxide (SiO₂) (also: silica, CAS No.: 7631-86-9), barium sulfate (BaSO₄) or iron oxide (Fe₂O₃). These inorganic surface coatings can occur on their own, in combination and/or in combination with organic coating materials.

Within the context of the present invention, organic surface coatings can consist of vegetable or animal aluminum stearate, vegetable or animal stearic acid, lauric acid, dimethylpolysiloxane (also: dimethicone), methylpolysiloxane (methicone), simethicone (a mixture of dimethylpolysiloxane with an average chain length of 200 to 350 dimethylsiloxane units and silica gel) or algininc acid. These organic surface coatings can occur on their own, in combination and/or in combination with inorganic coating materials.

The list of specified conventional UV filter substances which can be used for the purposes of the present invention is of course not intended to be limiting.

Particularly preferred UV filters are to be selected from the group octocrylene, homosalate, ethylhexyl salicylate (octyl salicylate), butylmethoxydibenzoylmethane, titanium dioxide, phenylbenzimidazolesulfonic acid, bis-ethylhexyloxyphenolmethoxyphenoltriazine (Tinosorb® S), polysilicone-15, diethylaminohydroxybenzoylhexylbenzoate, disodium phenyldibenzimidazoletetrasulfonate, terephthalidenecamphorsulfonic acid, ethylhexyltriazone, diethylhexylbutamidotriazone, 2-ethylhexyl methoxycinnamate, isoamyl p-methoxycinnamate, polysilicone-15, benzophenone-4, methylenebis-benzotriazolyltetramethylbutylphenol, titanium dioxide (with and without coating), zinc oxide (with and without coating), 2,4,6-tris(biphenyl)-1,3,5-triazine and benzophenone-3.

A high UV filter fraction means that the fraction of UV photoprotective substances constitutes more than 5% by weight, based on the total mass of the preparation. Preferably, the fraction is selected in the range from 10% by weight to 40% by weight, in particular between 20 and 35% by weight. For an SPF of the preparation of 30, the preparation advantageously comprises a UV filter substance fraction of 20 to 30% by weight, based on the total mass of the preparation.

For an SPF of the preparation of 50, the preparation advantageously comprises a UV filter substance fraction of 25 to 35% by weight, based on the total mass of the preparation.

The preparations furthermore advantageously comprise one or more skin moisturizers with a fraction up to 10% by weight, preferably between 2 and 7.5% by weight, particularly preferably between 2.5 and 5% by weight, based on the total mass of the preparation.

Skin moisture is a term from the cosmetics industry. Healthy skin has natural moisture. Only if the human skin has anomalies with regard to dryness does inadequate skin moisture come to bear. Apart from causes resulting from illness, the age of the human skin as well as the pigmentation also plays a role. An important role for the moisture of the skin is played by moisturizing factors such as e.g. urea. These can be supplied to the skin by skincare compositions.

Normally, the human skin requires no kind of auxiliaries for maintaining natural moisture. However, an unhealthy lifestyle, dry air (particularly in solariums and heated and/or air-conditioned inner rooms), environmental influences, stress and long-term sunbathing contribute to the removal of moisture. Long and hot baths and detergent residues in clothing also cause the loss of the important constituents of the hydro lipid system of the skin. In order to prevent the skin from drying out, moisturizing creams are used. Refatting soaps and largely soap-free detergents also return the fat back to the skin. As a result, it becomes smoother and more supple again. Here, body cleaning compositions which do not destroy the natural acid protective mantle of the skin (pH of the skin=5.5) are particularly recommended.

In some apartments, the aeration of which is not possible as usual as a result of negative environmental conditions, so-called humidifiers can also be used to assist healthy skin moisture.

Moisturizing agents, also termed moisturizers, is the term used to refer to substances or substance mixtures which impart to cosmetic or dermatological preparations the property, following application and/or spreading on the surface of the skin, of reducing the moisture release from the horny layer (also called transepidermal water loss (TEWL)) and/or of positively influencing the hydration of the horny layer. The latter is measured by corneometry, a method which is sufficiently known to the expert.

It was hitherto disadvantageous that a high fraction of moisturizing agents in preparations led to the care emulsions having an unacceptable consistency and a stickiness that was undesirable to the user.

The present invention also rectifies this situation on account of the polyglycerol-10 stearate according to the invention.

Consequently, as for the stickiness on account of film formers, here too an improvement in the sensory properties merely by virtue of using PG-10 stearate is observed.

Polyglyceryl-10 stearate can therefore be used as an emulsifier in photoprotective preparations comprising one or more UV filter substances and one or more film formers and/or one or more skin moisturizers for reducing the stickiness compared with the same preparations without polyglyceryl-10 stearate.

Advantageous moisturizers for the purposes of the present invention are, for example, glycerol, lactic acid and/or lactates, in particular sodium lactate, butylene glycol, propylene glycol, panthenol, biosaccharide gum-1, glycine soya, urea, urea derivatives, glycerylglucose, ethylhexyloxyglycerol, pyrrolidonecarboxylic acid and hyaluronic acid, hyaluronic acid derivatives, amino acids and corresponding derivatives, glucosamineglycans, honey. Furthermore, it is particularly advantageous to use polymeric moisturizers from the group of the water-soluble and/or water-swellable and/or water-gellable polysaccharides. A fucose-rich polysaccharide, which is listed in the Chemical Abstracts under the registry number 178463-23-5 and is available e.g. under the name Fucogel®1000 from SOLABIA S.A., is particularly advantageous. Moisturizers can advantageously also be used as antiwrinkle active ingredients for protecting against skin changes, as arise e.g. during skin aging.

Salts are also very good moisturizers. The latter can be selected for example from the group of the salts with chloride anions, also inorganic oxo element anions. Electrolytes based on organic anions are also advantageous, e.g. lactates, acetates, benzoates, citrates, amino acids, ethylenediaminetetraacetic acid and salts thereof and others besides. Cations of the salts used are preferably ammonium, alkali metal, alkaline earth metal, magnesium, iron and zinc ions. It goes without saying that only physiologically acceptable electrolytes should be used in cosmetics. Particular preference is given to cooking salt, sea salt, dead sea salt, magnesium sulfate, magnesium chloride, zinc sulfate and mixtures thereof

The preferred moisturizer is glycerol.

Further particularly preferred moisturizers are for example polyols.

Polyols is the term used to refer to polyhydric alcohols, i.e. organic compounds which carry at least 2 alcoholic hydroxyl groups in the molecule. In one embodiment of the invention, the polyols contain 2 to 6 hydroxyl groups per molecule. In one embodiment of the invention, the polyols used are low molecular weight polyhydric alcohols, i.e. compounds which contain 2 to 18, in particular 2 to 10, preferably 2 to 6, carbon atoms.

In a preferred embodiment of the invention, the polyols used are compounds which carry at least 2 hydroxyl groups per molecule and consist of 2 to 18, preferably 2 to 10, in particular 2 to 6, carbon atoms.

In a preferred embodiment of the invention, the polyols used are compounds which carry 2 to 6 hydroxyl groups per molecule and consist of 2 to 6 carbon atoms.

The polyols used are either individual polyols or mixtures of any desired polyols. The polyols can also contain further functional groups, in particular amino groups, and/or be modified with nitrogen. In a preferred embodiment, the polyols contain no further functional groups apart from the hydroxyl groups.

Typical examples of polyols to be used according to the invention are glycerol, diglycerol, triglycerol, tetraglycerol, alkylene glycols, such as, for example, ethylene glycol, diethylene glycol, triethylene glycol, 1,2-propylene glycol, 1,3-propylene glycol, butylene glycol, hexylene glycol, and polyethylene glycols with an average molecular weight from 100 to 1000 daltons; technical-grade oligoglycerol mixtures with a degree of intrinsic condensation of 1.5 to 10, such as, for example, technical-grade diglycerol mixtures with a diglycerol content from 40 to 50% by weight;

methylol compounds, such as in particular trimethylolethane, trimethylolpropane, trimethylolbutane, pentaerythritol and dipentaerythritol;
short-chain alkylglucosides, in particular those with 1 to 8 carbon atoms in the alkyl radical, such as, for example, methyl and butyl glucoside;
sugar alcohols with 5 to 12 carbon atoms, such as, for example, erythritol, arabitol, adonitol (synonym ribitol), xylitol, sorbite, sorbitol, mannitol and dulcite (synonym galactitol).

Sugars with 5 to 12 carbon atoms, such as, for example, glucose or sucrose;

amino sugars, such as, for example, glucamine; dialcohol amines, such as diethanolamine or 2-amino-1,3-propanediol.

1,2-Alkanediols, in particular 1,2-pentanediol, 1,2-hexanediol and 1,2-octanediol, are also advantageous skin moisturizers. The latter are preferably used in use concentrations in the range between 0.1 and 2% by weight, preferably 0.2 to 1% by weight, particularly preferably 0.3 to 0.75% by weight, based on the total mass of the preparation.

Application forms of the preparations according to the invention include e.g.: solutions, suspensions, emulsions, PIT emulsions, hydrodispersions, thin-liquid emulsion as impregnating agent (e.g. wipes), pastes, ointments, gels, creams, lotions, aerosols and sprays.

Preferably, the preparations according to the invention are O/W, W/O, W/O/W, O/W/O emulsions and hydrodispersions, where O can also stand for silicone oils. Preferably, the preparation is formulated on the basis of an O/W emulsion or hydrodispersion.

The preparations according to the invention advantageously comprise no parabens, formaldehyde donors, organohalic substances, benzoic acid and salts thereof, formates and tea tree oil, and advantageously also no benzyl alcohol and/or phenoxyethanol.

These substances are all customary preservatives, but also have disadvantages. For example, DMDM hydantoin is a formaldehyde donor. As is generally known, formaldehyde is a toxic substance and its use in cosmetics is not without controversy. Benzylalcohol has a characteristic odor which has to be masked with perfume in a formulation. Perfume ingredients are unfortunately often also suspected of having irritative or sensitizing effects. Consequently, it is desirable to keep the perfume concentration as low as possible. Phenoxyethanol is again suspected of triggering type IV contact allergies and is thus being increasingly shunned by consumers. Organic halides are comparatively highly reactive substances and interact with the cell membrane and are thus greatly shunned by consumers. Tea tree oil is not approved as a medicament and is classed as a risk substance for the appearance of contact dermatitides.

The cosmetic or dermatological preparations according to the invention can also comprise cosmetic auxiliaries and further active ingredients as are customarily used in such preparations, e.g. preservatives, preserving assistants, bactericides, substances for preventing foaming, dyes and colored pigments, thickeners, moisturizing and/or humectant substances, fats, oils, waxes or other customary constituents of a cosmetic or dermatological formulation such as alcohols, polyols, polymers, foam stabilizers, electrolytes, organic solvents or silicone derivatives, self-tanning agents, buffers, pH regulators, vegetable extracts, surfactants, propellant gases, powders, sebum-absorbing substances, UV filters, active ingredients such as, for example, antiage, anticellulite, antiacne, antirosacea, antineurodermatitis, antioxidants, moisturizers, chelate formers, antiperspirants, bleaches and colorants etc., provided the addition does not hinder the required properties as regards emulsifier content, required stability and in particular the water resistance.

Preferred lipid components in the preparations according to the invention are C 12-15 alkyl benzoate, isopropyl palmitate, caprylic/capric triglyceride and/or octyldodecanol.

When producing the preparations according to the invention, a further unexpected advantage is revealed when using polyglyceryl-10 stearate.

During emulsion preparation, normally a water phase and a fatty phase have to be heated and mixed, and this mixture then has to be cooled again. This means an at least duplicate addition of heat energy. In energetic terms, this is unfavorable and likewise problematic for any thermally labile active ingredients.

Surprisingly, this duplicate heating is not necessary as a result of using polyglyceryl-10 stearate as emulsifier.

Emulsion preparations with PG-10 stearate can be produced in the so-called cold-cold process. Normally, the fatty phase is melted at 80-100° C. and, in parallel, the water phase is heated to 80-100° C. These two hot phases are combined and stirred. Homogenization is then carried out. In the production, it is often necessary to cool for this purpose. This process is called hot/hot process. Cold/cold process thus means analogously that the water phase is at room temperature and the fatty phase is at room temperature, and these are combined and homogenized, which is a great advantage in energetic terms. The preparations according to the invention are therefore preferred for the incorporation of thermally labile active ingredients, such as, for example, natural oils, active ingredients, vitamins, perfume and also individual perfume ingredients.

Thermally labile is the term used to refer to those substances which, upon heating to above 50° C., change as regards color, odor or in respect of physical parameters, or even partly or completely break down.

The examples below illustrate the preparations according to the invention. The stated fractions are fractions by weight, in each case based on the total mass of the preparation.

### EXAMPLES

| O/W emulsion | 1 | 2 | 3 | 4 | 5 |
|---|---|---|---|---|---|
| Polyglyceryl-10 stearate | 2 | 2 | 1 | 0.5 | 0.2 |
| Glyceryl stearate SE | | 0.5 | 1 | 1 | 1 |
| Cetearyl alcohol | | | 1.5 | 1 | |
| Stearyl alcohol | 2 | 1.5 | | | |
| Acrylates/C₁₀₋₃₀ alkyl acrylate crosspolymer | | 0.2 | | | 0.1 |
| Xanthan gum | 0.4 | | 0.2 | 0.2 | 0.3 |
| C₁₂₋₁₅ Alkyl benzoate | | 3 | | | 5 |
| Dicaprylyl carbonate | | 2 | | | |
| Myristyl myristate | | | 2 | | 1 |
| Butylene glycol dicaprylate/dicaprate | | | 3 | | 3 |
| Propylheptyl caprylate | 5 | | 5 | 2 | |
| Dicaprylyl ether | | | | | 2 |
| Octyldodecanol | 1 | | | | |
| Cyclopentasiloxane | | 5 | 5 | 1 | 10 |
| MT propylsilsesquioxane wax resin with | 2 | 3 | 1 | 5 | 1.5 |
| M = Si(C30+)(CH₃)₂ | | | | | |
| Dimethicone | 6 | | | 5 | |
| Dimethiconol | | 5 | | | |
| 1-Methyl-1,3-propanediol | 5 | 8 | | | |
| Glycerol | 3 | 5 | 3 | 5 | 3 |
| Octane-1,2-diol | 2 | | 1 | | |
| Bisvinyldimethicone/PPG-20 crosspolymer | | 4 | 1 | | |
| Hexyl 2-(4′-diethylamino-2′-hydoxybenzoyl)benzoate | 3 | 5 | 1 | 0.5 | 2 |
| Octocrylene | | | 5 | | |
| Titanium dioxide | | 0.5 | 1 | | 2 |
| Phenylbenzimidazole sulfonic acid | | | | 4 | 2 |
| Octyl salicylate | | | 5 | | |
| Polyisilicone-15 | | | | | 2 |
| Ethylhexyl methoxycinnamate | | 10 | | | |
| Methylenebisbenzotriazolyl tetramethylbutylphenol | 3 | | | 2 | |
| Ethylhexyltriazone | 3 | 2 | | | 3 |
| Tristriphenyltriazine | | | 2 | | |
| Bisethylhexylphenol methoxyphenyltriazine | | 2 | | | |
| PVP/hexadecene copolymer | | 0.5 | 0.1 | | |
| Vitamin E acetate | 0.2 | 0.2 | 0.2 | 0.3 | 0.1 |
| Na₂H₂EDTA | 0.1 | 0.1 | | | 0.2 |
| Perfume | 0.2 | 0.3 | 0.3 | 0.4 | 0.25 |
| Methylisothiazolinone | 0.05 | 0.1 | 0.05 | 0.1 | 0.6 |
| Ethylhexylglycerol | 0.25 | 0.25 | 0.5 | 0.5 | |
| Benzyl alcohol | | | 0.5 | | 0.5 |
| Pentanediol | 1 | | | 0.5 | 1.5 |
| Methylpropanediol | | 3 | | | 3.5 |
| Sodium hydroxide | q.s. | q.s. | q.s. | q.s. | q.s. |
| Water | ad 100.0 | ad 100.0 | ad 100.0 | ad 100.0 | ad 100.0 |
| | 6 | 7 | 8 | 9 | 10 |
| Glyceryl stearate SE | 2 | 0.5 | 3 | 0.5 | 0.2 |
| PG-10 stearate | 1.5 | 0.5 | 0.1 | 1.0 | 1.5 |
| Polyglyceryl-3 methylglycose distearate | | | | 0.5 | 2.5 |
| Copolymer of vinylpyrrolidone and acrylic acid | | 1.5 | | | |
| Stearyl alcohol | | | | 2 | |
| Cetyl alcohol | 2 | | | | 2 |
| Acrylates/C₁₀₋₃₀ alkyl acrylate crosspolymer | | | 0.3 | 0.2 | 0.1 |
| Carbomer | | | 0.2 | | 0.1 |
| Xanthan gum | 0.3 | | | | |
| C₁₂₋₁₅ Alkyl benzoate | 3 | 5 | | | |
| Butylene glycol dicaprylate/dicaprate | | | 5 | 7 | |
| Dicaprylyl carbonate | | 2 | | 2 | |
| Octyldodecanol | | | | | 2 |
| Cyclic silicone | | 2 | 10 | 5 | |
| MT propylsilsesquioxane wax resin with | 2 | | | 5 | 3 |
| M = Si(C 30+)(CH₃)₂ | | | | | |
| Linear silicone | 5 | 8 | 5 | | |
| Aluminum starch octenylsuccinate | | 0.5 | 1 | | |
| Glycerol | 2 | 4 | 5 | 10 | 8 |
| Ethanol | 3 | 4 | | | 4 |
| Hexyl 2-(4′-diethylamino-2′-hydoxybenzoyl)benzoate | | 1.5 | | 0.5 | 6 |
| Octocrylene | 2.5 | | 6 | 5 | 7.5 |
| Butylmethoxy dibenzoylmethane | 2.5 | | 3 | 2 | |
| Ethylhexyl methoxy cinnamate | | | | | 7.5 |
| Octyl salicylate | | 5 | | | |
| Phenylbenzimidazole sulfonic acid | 1 | 1 | | | |
| Titanium dioxide | | | 5 | | 3 |
| Bisethylhexylphenol methoxyphenyltriazine | | 2 | | | 2 |
| Isoamyl methoxycinnamate | 5 | | | | |
| Ethylhexyl triazine | | | | 2 | 2 |
| Terephthalyidene dicamphorsulfonic acid | | 4 | | | |
| Vitamin E acetate | 0.2 | 0.1 | 0.5 | 0.25 | 0.3 |
| Na₂H₂EDTA | 0.2 | 0.2 | 0.2 | 0.2 | 0.5 |
| Starch | 1 | | | 3 | |
| Perfume | | 0.2 | 0.1 | 0.3 | 0.25 |
| Methylparaben | 0.4 | 0.3 | | 0.2 | 0.4 |
| Ethylparaben | 0.4 | 0.3 | | | |
| Phenoxyethanol | 0.5 | 0.7 | 0.5 | | |
| Methylisothiazolinone | | | 0.5 | 0.5 | |
| Methylpropanediol | | | | | 3 |
| Caprylyl glycol | | | | | 0.25 |
| Sodium hydroxide, dyes | q.s. | q.s. | q.s. | q.s. | q.s. |
| Water | ad 100 | ad 100 | ad 100 | ad 100 | ad 100 |
| | 11 | 12 | 13 | 14 | 15 |
| Glyceryl stearate | 0.2 | 2 | 0.75 | 1 | 0.5 |
| PG-10 stearate | 2 | 0.25 | 1 | 1 | 1.25 |
| Cetearyl alcohol | 4 | | | 2 | |
| Stearyl alcohol | | 2 | 1 | | |
| Cetyl alcohol | | | 1 | 1 | |
| Acrylates/C₁₀₋₃₀ alkylacrylate crosspolymer | | | 0.05 | 0.2 | 0.2 |
| Carbomer | 0.1 | | 0.2 | | |
| Xanthan gum | | 0.3 | | | |
| Triheptanoin | | | 2 | | |
| C₁₂₋₁₅ Alkyl benzoate | | 7 | | | 3 |
| Butylene glycol dicaprylate/dicaprate | 2 | 4 | | | 5 |
| Dicaprylyl carbonate | 4 | | | | |
| Phenylethyl benzoate | | | 5 | 5 | 4 |
| Diisopropyl sebacate | | 3 | | 5 | |
| Cyclic silicone | 3 | | | | |
| 2-Propylheptyl octanoate | 4 | | 3 | | 2 |
| MT propylsilsequioxane wax resin with | 2 | | 8 | | 3 |
| M = Si(C 30+)(CH₃)₂ | | | | | |
| Glycerol | 7.5 | 5 | 10 | 3 | 5 |
| Ethanol | | | 2 | | 4 |
| Bisethylhexyloxyphenol methoxyphenyltriazine | 2 | 3 | 0.5 | 1.5 | 3 |
| Ethylhexyl methoxycinnamate | | | 7 | 1 | 4 |
| Octyl salicylate | | | | | 5 |
| Homosalate | | | 3 | | |
| Octocrylene | 5 | | | 4 | |
| Phenylbenzimidazole sulfonic acid | 1 | | | 2 | |
| Butylmethoxy dibenzolmethane | 2 | | 3 | 4 | 1 |
| 2,4,6-Tris(biphenyl)-1,3,5-triazine | | 2 | | | 1 |
| Diethylhexylbutamidotriazine | | | 2 | 1.5 | |
| Ethylhexyltriazine | | | | 1.5 | |
| Drometrizole trisiloxane | | 2 | | | |
| Tapioca starch | 1 | | 2.5 | | |
| Sodium starch octenylsuccinate | | | | 1 | |
| Na₂H₂EDTA | 0.1 | | | | |
| Perfume | 0.2 | 0.3 | 0.4 | 0.5 | |
| Parabens | 0.5 | | | | |
| Sodium hydroxide, dyes | q.s. | q.s. | q.s. | q.s. | q.s. |
| Ethylhexyl glycerol | | | 1 | 0.5 | 1 |
| Caprylyl glycol | | 1 | | 0.5 | 1 |
| Ethyllauroylarguiate 20% in glycerol | 1.8 | 1.8 | 1.8 | 1.8 | 0.05 |
| Phenoxyethanol | 0.5 | 0.5 | 0.5 | 0.5 | |
| Water | ad 100.0 | ad 100.0 | ad 100.0 | ad 100.0 | ad 100.0 |
| | 16 | 17 | 18 | 19 | 20 |
| Glyceryl stearate | 0.2 | 2 | 1 | 2 | 1 |
| PG-10 stearate | 2 | 0.25 | 1 | 0.25 | 1 |
| Cetearyl alcohol | 4 | | 2 | | 2 |
| Stearyl alcohol | | 2 | | 2 | |
| Cetyl alcohol | | | 1 | | 1 |
| Acrylates/C₁₀₋₃₀ alkyl acrylate crosspolymer | | | 0.2 | | 0.2 |
| Carbomer | 0.1 | | | | |
| Xanthan gum | | 0.3 | | 0.3 | |
| Triheptanoin | | | | | |
| C₁₂₋₁₅ Alkyl benzoate | | 7 | | 7 | |
| Butylene glycol dicaprylate/dicaprate | 2 | 4 | | 4 | |
| Dicaprylyl carbonate | 4 | | | | |
| Phenylethyl benzoate | | | 5 | | 5 |
| Diisopropyl sebacate | | 3 | 5 | 3 | 5 |
| Cyclic silicone | 3 | | | | |
| 2-Propylheptyl octanoate | 4 | | | | |
| MT propylsilsequioxane wax resin with | 2 | | | | |
| M = Si(C 30+)(CH₃)₂ | | | | | |
| Glycerol | 7.5 | 5 | 3 | 5 | 3 |
| Ethanol | | | 3 | 3 | |
| Ethylhexyl methoxycinnamate | 7.00 | 7.00 | 7.00 | 7.00 | 7.00 |
| Octocrylene | 2.80 | 2.80 | 2.80 | 2.80 | 2.80 |
| Butylmethoxy dibenzoylmethane | 3.00 | 3.00 | 3.00 | 3.00 | 3.00 |
| Benzotriazolyldodecyl p-cresol | 9.00 | | | | |
| Diethylhexyl 2,6-naphthalate | | 9.00 | | | 4.5 |
| Zinc oxide | | | 5 | | 25 |
| Ethylmethylmethoxycrylene | | | | 6 | |
| Titanium dioxide | | | | 5 | 5 |
| Silica | | | 0.5 | | |
| Na₂H₂EDTA | 0.1 | | | | |
| Perfume | 0.2 | 0.3 | 0.5 | 0.3 | 0.5 |
| Sodium hydroxide, dyes | q.s. | q.s. | q.s. | q.s. | q.s. |
| Ethylhexylglycerol | 1 | | | | |
| Caprylyl glycol | | 1 | 0.5 | 1 | 0.5 |
| Piroctone olamine | | | 0.05 | | 0.05 |
| Phenoxyethanol | 0.5 | | | | |
| Water | ad 100.0 | ad 100.0 | ad 100.0 | ad 100.0 | ad 100.0 |

1.-13. (canceled)

14. A water-resistant cosmetic or dermatological photoprotective preparation, wherein the preparation comprises one or more UV filter substances, less than 6% by weight of polyglyceryl-10 stearate, based on a total weight of the preparation, and comprises no lecithins, O/W emulsion preparations of the following compositions I to IV being excluded:

| Composition | I | II |
|---|---|---|
| Panthenol | 0.7 | 0 |
| Butylene glycol | 5 | 10 |
| Propylene glycol | 5 | — |
| Benzethonium chloride | 0.5 | 0.9 |
| Lauroyl ethyl arginate | 1.0 | 0.5 |
| Caprylic/capric triglyceride | 0 | 1.00 |
| Cetearyl alcohol | 2.5 | 4.00 |
| Linear silicone oil | 0 | 0.50 |
| C12-15 Alkyl benzoate | 0 | 2.00 |
| Dicaprylyl ether | 10 | 0 |
| Glyceryl stearate | 2.4 | 2.4 |
| Glycerol | 8 | 8 |
| Butylene glycol | 1 | 0 |
| Sodium hydroxide solution 45% | 0.25 | 1 |
| Polyglyceryl-10 stearate | 1 | 1 |
| Acrylic acid/VP crosspolymer | 0.5 | 0.75 |
| Trisodium EDTA | | 1 |
| Ethylhexyl methoxycinnamate | 2 | 0 |
| Butylmethoxy dibenzoylmethane | 0 | 2 |
| Phenylbenzimidazole sulfonic acid, sodium salt | 0 | 2 |
| Ethylhexyl salicylate | 0 | 2 |
| Titanium dioxide, silicone-coated | 0.3600 | 0 |
| Octocrylene | 0 | 2 |
| Perfume | 0 | 0.20 |
| Water | ad 100 | ad 100 |
| Composition | III | IV |
| Panthenol | 0.7 | 0 |
| Butylene glycol | 5 | 10 |
| Propylene glycol | 5 | — |
| Methylisothiazolinones | 0.06 | — |
| Benzethonium chloride | 0.15 | — |
| Piroctone olamine | 0.15 | — |
| Lauroyl ethyl arginate | 0.15 | 1.75 |
| Caprylic/capric triglyceride | 0 | 1.00 |
| Cetearyl alcohol | 2.5 | 4.00 |
| Linear silicone oil | 0 | 0.50 |
| C12-15 Alkyl benzoate | 0 | 2.00 |
| Dicaprylyl ether | 10 | 0 |
| Glyceryl stearate | 2.4 | 2.4 |
| Glycerol | 8 | 8 |
| Sodium hydroxide solution 45% strength | 0.25 | 1 |
| Polyglyceryl-10 stearate | 1 | 1 |
| Acrylic acid/VP crosspolymer | 0.5 | 0.75 |
| Trisodium EDTA | | 1 |
| Ethylhexyl methoxycinnamate | 2 | 0 |
| Butylmethoxy dibenzoylmethane | 0 | 2 |
| Phenylbenzimidazole sulfonic acid | 0 | 2 |
| Ethylhexyl salicylate | 0 | 2 |
| Titanium dioxide + trimethoxycaprylylsilane | 0.3600 | 0 |
| Octocrylene | 0 | 2 |
| Perfume | 0 | 0.20 |
| Water | ad 100 | ad 100 |

numerical values representing fractions by weight, based on a total mass of the preparation.

15. The preparation of claim 14, wherein the preparation further comprises one or more film formers.

16. The preparation of claim 14, wherein the preparation comprises from 0.1% to 2.8% by weight of polyglyceryl-10 stearate

17. The preparation of claim 14, wherein apart from polyglycerol-10 stearate, the preparation comprises no further emulsifiers.

18. The preparation of claim 16, wherein apart from polyglycerol-10 stearate, the preparation comprises no further emulsifiers.

19. The preparation of claim 18, wherein the preparation further comprises one or more film formers.

20. The preparation of claim 14, wherein in addition to polyglycerol-10 stearate, the preparation further comprises one or more non-ethoxylated emulsifiers which are solid, pasty or liquid at 25° C.

21. The preparation of claim 20, wherein the one or more non-ethoxylated emulsifiers comprise glyceryl stearate.

22. The preparation of claim 16, wherein in addition to polyglycerol-10 stearate, the preparation further comprises one or more non-ethoxylated emulsifiers which are solid, pasty or liquid at 25° C.

23. The preparation of claim 22, wherein the one or more non-ethoxylated emulsifiers comprise glyceryl stearate.

24. The preparation of claim 14, wherein the preparation is present as an O/W emulsion.

25. The preparation of claim 14, wherein the preparation is present as a hydrodispersion.

26. The preparation of claim 14, wherein the one or more UV filter substances comprise one or more of octocrylene, homosalate, ethylhexyl salicylate (octyl salicylate), butylmethoxy dibenzoylmethane, titanium dioxide, phenylbenzimidazole sulfonic acid, bisethylhexyloxyphenol methoxyphenyltriazine, polysilicone-15, diethylamino-hydroxybenzoyl hexylbenzoate, disodium phenyldibenzimidazole tetrasulfonate, terephthalidene camphorsulfonic acid, ethylhexyltriazone, diethylhexylbutamidotriazone, 2-ethylhexyl methoxycinnamate, isoamyl p-methoxycinnamate, benzophenone-4, methylenebisbenzotriazolyltetramethylbutylphenol, titanium dioxide (with and without coating), zinc oxide (with and without coating), 2,4,6-tris(biphenyl)-1,3,5-triazine, and benzophenone-3.

27. The preparation of claim 14, wherein the preparation further comprises one or more skin moisturizers.

28. The preparation of claim 14, wherein the preparation is free of parabens, formaldehyde donors, organohalic substances, benzoic acid and salts thereof, formats, and tea tree oil.

29. The preparation of claim 16, wherein the preparation is free of parabens, formaldehyde donors, organohalic substances, benzoic acid and salts thereof, formats, and tea tree oil.

30. The preparation of claim 18, wherein the preparation is free of parabens, formaldehyde donors, organohalic substances, benzoic acid and salts thereof, formats, and tea tree oil.

31. The preparation of claim 20, wherein the preparation is free of parabens, formaldehyde donors, organohalic substances, benzoic acid and salts thereof, formats, and tea tree oil.

32. A method of improving the water resistance of a photoprotective preparation comprising one or more UV filter substances, wherein the method comprises incorporating in the preparation polyglyceryl-10 stearate.

33. A method of improving the stickiness of a photoprotective preparation comprising one or more UV filter substances and one or more film formers and/or one or more skin moisturizers, wherein the method comprises incorporating in the preparation polyglyceryl-10 stearate.
